(19) United States
(12) Patent Application Publication
(10) Pub. No.: US 2004/0009129 A1
(43) Pub. Date: Jan. 15, 2004

(54) Composition for refreshing oral cavity by removal of oral smell

(76) Inventor: Joo Hwan Yang, Bucheon (KR)

Correspondence Address: JACOBSON HOLMAN PLLC, 400 SEVENTH STREET N.W., SUITE 600, WASHINGTON, DC, 20004, US

(21) Appl. No.: 10/382,906

(22) Filed: Mar. 7, 2003

(30) Foreign Application Priority Data
Jul. 12, 2002 (KR) 2002-40624

(51) Int. Cl. A61K 7/16

(52) U.S. Cl. 424/49

(57) ABSTRACT

The present invention relates to a composition for refreshing oral cavity comprising i) carbonate candy, sugar alcohol or mixture of them as base material, ii) one or more refreshing functional agent selected from the group consisting of silicon dioxide, sodium silicoaluminate, calcium carbonate and silica, iii) artificial sweetener, iv) nutrient component selected from vitamin or mineral, v) natural or artificial flavor, and vi) coloring agent. Further, the composition of the present invention has the merits of convenience of portable storage and use.

### BACKGROUND OF THE INVENTION

The present invention relates to a composition for refreshing oral cavity by removal of oral smell. More particularly, this invention relates to a composition for refreshing oral cavity, which is filled in the capsule or sealed packing for convenient use.

The composition for refreshing oral cavity comprises i) carbonate candy, sugar alcohol or mixture of them as base material, ii) one or more refreshing functional agent selected from the group consisting of silicon dioxide, sodium silicoaluminate, calcium carbonate and silica, iii) artificial sweetener, iv) nutrient component selected from vitamin or mineral, v) natural or artificial flavor, and vi) coloring agent. Further, the composition of the present invention has the merits of convenience of portable storage and use.

For refreshing and cleaning the oral cavity, many kinds of liquid oral cleansers have been commercially marketed. For example, Care Gargle™ containing benzetonium chloride, Gargreen Mint™ containing CPC and xylitol, Pro Fresh™ containing polyphenols and aloe, Gargreen Tent™ containing xylitol, Retradex™ containing aloe vera, chlorophyll and mint flavor and oral cleanser containing cinnamon extract have been developed and marketed. As the packings of such liquid oral cleanser, bottle packing or pouch pack has been developed.

On the other hand, some candies for removing oral smell and chewing gums having various flavors or oral cleaning agent, such as, cethyl pyridium chloride have been also developed and commercially marketed.

However, such liquid oral cleanser has some drawbacks in use, because it has large volume and weight. Further, it requires additional cup and bottle for using it, which causes the inconvenience of use. Of course, the gargling the liquid oral cleanser and throwing it out from oral cavity are very inconvenient for user.

Therefore, there has been a need for developing new type of oral cleanser, which can be portably used in a small amount for one or daily use without additional devices, for example, cups or bottles for solving said drawbacks.

To meet above requirements, the inventor of the present invention has developed a powder type of oral refreshing agent which can be filled in the capsules. Further, the oral refreshing agent of the present invention has many merits; i) convenient use of agent by dropping it down to the oral cavity in a small amount, ii) portable storage for one or daily amount, iii) no need of additional device for use, for example, cup or bottle.

### SUMMARY OF THE INVENTION

The object of the present invention is to provide a composition for refreshing oral cavity by removal of oral smell comprising i) 30˜90 wt % of base material selected from carbonate candy, sugar alcohol or mixture of them, ii) 1˜20 wt % of refreshing functional agent one or more selected from the group consisting of silicon dioxide, sodium silicoaluminate, calcium carbonate and silica, iii) 0.01˜10 wt % of artificial sweetener, iv) 0.1˜30 wt % of nutrient component selected from vitamin or mineral, v) 0.1˜40 wt % of natural or artificial flavor and vi) a small amount of coloring agent.

Another object of the present invention is to provide a capsule or sealed packing filled with said composition for refreshing oral cavity by removal of oral smell.

In the present invention, said artificial sweetener is one or more selected from the group consisting of saccharine, aspartam, stevioside and persimmonic acid. Further, said natural or artificial flavor is one or more selected from the group consisting of orange flavor, strawberry flavor, grape flavor, peppermint flavor, spearmint flavor, menthol, anenol, oegenol, wintergreen flavor, mint flavor, sage, eucalyptus flavor, methyl salicylate, fruit extract, rosemary, green tea extract, persimmon extract and shanpinion extract.

### DETAILED DESCRIPTION OF THE INVENTION

The removal of oral smell is very important for social relationship, because the oral smell gives bad influence to face-to-face relationship as well as etiquette. Further, refreshing oral cavity is good for personal hygiene.

The present invention is focused to refreshing oral cavity and removing oral smell after having a meal or smoking the cigaret. The refreshing composition of the present invention is dissolved in the mouth, and the carbonate is generated after dissolution. Of course, the sugar alcohol can protect the teeth and flavoring agent gives a fresh flavor to all oral cavity as well as the removal of oral smell, which can not be accomplished by commercial liquid or solid type oral cleanser.

The present invention can be explained more concretely by following examples, but the scope of the present invention shall not be limited by following examples.

### EXAMPLE 1

Preparation of Composition for Refreshing Oral Cavity

Natural pigment is dissolved with distilled water, and make 3.5 wt % solution. This solution is absorbing to the base material, for example, carbonate candy powder or sugar alcohol powder. The colored base material powder is dried at 60° C. for 2 hours with warm wind.

8 g of refreshing functional agent one or more selected from the group consisting of silicon dioxide, sodium silicoaluminate, calcium carbonate and silica is mixed with 60 g of colored base material powder. Then, 10 g of nutrient component selected from vitamin or mineral; 4 g of at least one artificial sweetener selected from saccharine, aspartam, stevioside and persimmonic acid; and 18 g of at least one natural or artificial flavor selected from orange flavor, strawberry flavor, grape flavor, peppermint flavor, spearmint flavor, menthol, anenol, oegenol, wintergreen flavor, mint flavor, sage, eucalyptus flavor, methyl salicylate, fruit extract, rosemary, green tea extract, persimmon extract and shanpinion extract are added and mixed with said mixture. Finally, the powder composition for refreshing oral cavity with removal of oral smell is made, and fill it to the capsule.

### EXAMPLE 2˜10

The example 2˜10 are made as the same manner of example 1 except that the amount of components are varied as following Table 1. Table 1 shows the amount of each components.

TABLE 1

| | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|
| base material(g) | 60 | 58 | 59 | 59 | 67 | 71 | 70 | 41 | 39 |
| refreshing | 8 | 9 | 8 | 9 | 7 | 8 | 7 | 9 | 8 |
| functional agent(g) | | | | | | | | | |
| artificial | 4 | 4 | 4 | 3 | 5 | 4 | 3 | 3 | 4 |
| sweetener(g) | | | | | | | | | |
| nutrient | 10 | 11 | 12 | 13 | 7 | 4 | 8 | 20 | 21 |
| component(g) | | | | | | | | | |
| natural or artificial | 18 | 18 | 17 | 16 | 14 | 13 | 12 | 27 | 28 |
| flavor(g) | | | | | | | | | |
| Total(g) | 100 | 100 | 100 | 100 | 100 | 100 | 100 | 100 | 100 |

### EXAMPLE 11

Functional Test

The functional test between a capsule filled with composition for refreshing oral cavity prepared by above example 1 and a commercially marketed liquid type oral cleanser has been carried out. Followings are protocol and result.

1) Selection of Panel

Total 10 well-trained panels with 5 men and 5 women (age: 25˜30) are selected.

2) Method

By lapse of 20 minutes after having a lunch or smoking a cigaret, a capsule prepared in the present invention and a commercially marketed liquid type oral cleanser are used for functional test. The test has been carried out for 5 days by repeating the test in the same manners.

3) Result

The result is indicated by the numbers, on condition that the average point is 5, the worst point is 1 and the best point is 10.

(Score: the worst←1 2 3 4 5 6 7 8 9 10→the best)

The Table 2 shows the result of functional test.

TABLE 2

| | the powder or capsule of the | | | | commercially marketed liquid | | | |
|---|---|---|---|---|---|---|---|---|
| | present invention | | | | type of oral cleanser | | | |
| panel | removal | chewing | taste and | | removal | chewing | taste and | |
| No. | of smell | feeling | flavor | convenience | of small | feeling | flavor | convenience |
| 1 | 8 | 9 | 9 | 9 | 5 | 6 | 5 | 5 |
| 2 | 7 | 9 | 9 | 9 | 6 | 7 | 5 | 5 |
| 3 | 8 | 8 | 9 | 9 | 7 | 6 | 6 | 6 |
| 4 | 9 | 9 | 9 | 8 | 6 | 7 | 6 | 5 |
| 5 | 8 | 9 | 7 | 9 | 6 | 6 | 6 | 5 |
| 6 | 8 | 9 | 9 | 9 | 6 | 7 | 5 | 5 |
| 7 | 7 | 8 | 8 | 7 | 7 | 7 | 6 | 6 |
| 8 | 8 | 8 | 9 | 9 | 5 | 5 | 6 | 6 |
| 9 | 9 | 8 | 9 | 9 | 5 | 5 | 6 | 5 |
| 10 | 8 | 9 | 9 | 8 | 5 | 7 | 6 | 5 |
| Total | 80 | 86 | 87 | 86 | 58 | 63 | 57 | 53 |

As showing Table 2, the capsule or powder of the present invention shows better functional test results in all fields of functional test point.

Therefore, the oral refreshing capsule or powder of the present invention has many merits compared to conventional liquid type of oral cleanser in many fields for example, i) better taste and flavor, ii) convenient use of agent by dropping it down to the oral cavity in a small amount, iii) portable storage for one or daily amount, iv) no need of additional device for use, for example, cup or bottle.

What is claimed is:

1. A composition for refreshing oral cavity by removal of oral smell comprising
i) 30˜90 wt % of base material selected from carbonate candy, sugar alcohol or mixture of them;
ii) 1˜20 wt % of refreshing functional agent one or more selected from the group consisting of silicon dioxide, sodium silicoaluminate, calcium carbonate and silica;
iii) 0.01˜10 wt % of artificial sweetener;
iv) 0.1˜30 wt % of nutrient component selected from vitamin or mineral;
v) 0.1˜40 wt % of natural or artificial flavor; and
vi) a small amount of coloring agent.

2. The composition for refreshing oral cavity by removal of oral smell according to claim 1, wherein said artificial sweetener is one or more selected from the group consisting of saccharine, aspartam, stevioside and persimmonic acid.

3. The composition for refreshing oral cavity by removal of oral smell according to claim 1, wherein said natural or artificial flavor is one or more selected from the group consisting of orange flavor, strawberry flavor, grape flavor, peppermint flavor, spearmint flavor, menthol, anenol, oegenol, wintergreen flavor, mint flavor, sage, eucalyptus flavor, methyl salicylate, fruit extract, rosemary, green tea extract, persimmon extract and shanpinion extract.

4. A capsule or sealed packing filled with said composition for refreshing oral cavity by removal of oral smell of claim 1.
